(19) United States
(12) Patent Application Publication
(10) Pub. No.: US 2007/0005080 A1
(43) Pub. Date: Jan. 4, 2007

(54) Bolt action fastener delivery assembly

(75) Inventors: Raymond Michael Wolniewicz III, Redmond, WA (US); Darren Crow, Mercer Island, WA (US)

Correspondence Address: GRAYBEAL JACKSON HALEY LLP, Suite 350, 155-108th Avenue N.E., Bellevue, WA, 98004-5973, US

(73) Assignee: EsophyX, Inc.

(21) Appl. No.: 11/172,428

(22) Filed: Jun. 29, 2005

(51) Int. Cl. A61B 17/10

(52) U.S. Cl. 606/142

(57) ABSTRACT

An assembly that delivers tissue fasteners for deployment in tissue comprises a stylet that guides a fastener into tissue. The stylet has a proximal end. The assembly further includes a bolt attached to the proximal end of the stylet, a receiver that slidingly receives the bolt permitting linear movement of the bolt and stylet along a path. The assembly may further comprise a pusher that intersects the path of the stylet and pushes the fastener along the stylet towards and into the tissue.

### FIELD OF THE INVENTION

The present invention generally relates to tissue fixation devices, and more particularly to devices for treating gastroesophageal reflux disease using the same. The present invention more particularly relates to a bolt action assembly that delivers such tissue fixation devices in surgical environments.

### BACKGROUND

Gastroesophageal reflux disease (GERD) is a chronic condition caused by the failure of the anti-reflux barrier located at the gastroesophageal junction to keep the contents of the stomach from splashing into the esophagus. The splashing is known as gastroesophageal reflux. The stomach acid is designed to digest meat, and will digest esophageal tissue when persistently splashed into the esophagus.

A principal reason for regurgitation associated with GERD is the mechanical failure of a deteriorated gastroesophageal flap to close and seal against high pressure in the stomach. Due to reasons including lifestyle, a Grade I normal gastroesophageal flap may deteriorate into a malfunctioning Grade III or absent valve Grade IV gastroesophageal flap. With a deteriorated gastroesophageal flap, the stomach contents are more likely to be regurgitated into the esophagus, the mouth, and even the lungs. The regurgitation is referred to as “heartburn” because the most common symptom is a burning discomfort in the chest under the breastbone. Burning discomfort in the chest and regurgitation (burping up) of sour-tasting gastric juice into the mouth are classic symptoms of gastroesophageal reflux disease (GERD). When stomach acid is regurgitated into the esophagus, it is usually cleared quickly by esophageal contractions. Heartburn (backwashing of stomach acid and bile onto the esophagus) results when stomach acid is frequently regurgitated into the esophagus and the esophageal wall is inflamed.

Complications develop for some people who have GERD. Esophagitis (inflammation of the esophagus) with erosions and ulcerations (breaks in the lining of the esophagus) can occur from repeated and prolonged acid exposure. If these breaks are deep, bleeding or scarring of the esophagus with formation of a stricture (narrowing of the esophagus) can occur. If the esophagus narrows significantly, then food sticks in the esophagus and the symptom is known as dysphagia. GERD has been shown to be one of the most important risk factors for the development of esophageal adenocarcinoma. In a subset of people who have severe GERD, if acid exposure continues, the injured squamous lining is replaced by a precancerous lining (called Barrett's Esophagus) in which a cancerous esophageal adenocarcinoma can develop.

Other complications of GERD may not appear to be related to esophageal disease at all. Some people with GERD may develop recurrent pneumonia (lung infection), asthma (wheezing), or a chronic cough from acid backing up into the esophagus and all the way up through the upper esophageal sphincter into the lungs. In many instances, this occurs at night, while the person is in a supine position and sleeping. Occasionally, a person with severe GERD will be awakened from sleep with a choking sensation. Hoarseness can also occur due to acid reaching the vocal cords, causing a chronic inflammation or injury.

GERD never improves without intervention. Life style changes combined with both medical and surgical treatments exist for GERD. Medical therapies include antacids and proton pump inhibitors. However, the medical therapies only mask the reflux. Patients still get reflux and perhaps emphysema because of particles refluxed into the lungs. Barrett's esophagus results in about 10% of the GERD cases. The esophageal epithelium changes into tissue that tends to become cancerous from repeated acid washing despite the medication.

Several open laparotomy and laproscopic surgical procedures are available for treating GERD. One surgical approach is the Nissen fundoplication. The Nissen approach typically involves a 360-degree wrap of the fundus around the gastroesophageal junction. The procedure has a high incidence of postoperative complications. The Nissen approach creates a 360-degree moveable flap without a fixed portion. Hence, Nissen does not restore the normal movable flap. The patient cannot burp because the fundus was used to make the repair, and may frequently experience dysphagia. Another surgical approach to treating GERD is the Belsey Mark IV (Belsey) fundoplication. The Belsey procedure involves creating a valve by suturing a portion of the stomach to an anterior surface of the esophagus. It reduces some of the postoperative complications encountered with the Nissen fundoplication, but still does not restore the normal movable flap. None of these procedures fully restores the normal anatomical anatomy or produces a normally functioning gastroesophageal junction. Another surgical approach is the Hill repair. In the Hill repair, the gastroesophageal junction is anchored to the posterior abdominal areas, and a 180-degree valve is created by a system of sutures. The Hill procedure restores the moveable flap, the cardiac notch and the Angle of His. However, all of these surgical procedures are very invasive, regardless of whether done as a laproscopic or an open procedure.

New, less surgically invasive approaches to treating GERD involve transoral endoscopic procedures. One procedure contemplates a machine device with robotic arms that is inserted transorally into the stomach. While observing through an endoscope, an endoscopist guides the machine within the stomach to engage a portion of the fundus with a corkscrew-like device on one arm. The arm then pulls on the engaged portion to create a fold of tissue or radial plication at the gastroesophageal junction. Another arm of the machine pinches the excess tissue together and fastens the excess tissue with one pre-tied implant. This procedure does not restore normal anatomy. The fold created does not have anything in common with a valve. In fact, the direction of the radial fold prevents the fold or plication from acting as a flap of a valve.

Another transoral procedure contemplates making a fold of fundus tissue near the deteriorated gastroesophageal flap to recreate the lower esophageal sphincter (LES). The procedure requires placing multiple U-shaped tissue clips around the folded fundus to hold it in shape and in place.

This and the previously discussed procedure are both highly dependent on the skill, experience, aggressiveness, and courage of the endoscopist. In addition, these and other procedures may involve esophageal tissue in the repair. Esophageal tissue is fragile and weak, in part due to the fact, that the esophagus is not covered by serosa, a layer of very sturdy, yet very thin tissue, covering and stabilizing all intraabdominal organs, similar like a fascia covering and stabilizing muscle. Involvement of esophageal tissue in the repair of a gastroesophageal flap valve poses unnecessary risks to the patient, such as an increased risk of fistulas between the esophagus and the stomach.

A new and improved apparatus and method for restoration of a gastroesophageal flap valve is fully disclosed in U.S. Pat. No. 6,790,214, is assigned to the assignee of this invention, and is incorporated herein by reference. That apparatus and method provides a transoral endoscopic gastroesophageal flap valve restoration. A longitudinal member arranged for transoral placement into a stomach carries a tissue shaper that non-invasively grips and shapes stomach tissue. A tissue fixation device is then deployed to maintain the shaped stomach tissue in a shape approximating a gastroesophageal flap.

Whenever tissue is to be maintained in a shape as, for example, in the improved assembly last mentioned above, it is necessary to fasten at least two layers of tissue together. In applications such as gastroesophageal flap valve restoration, there is very limited room to maneuver a fastener deployment device. For example, this and other medical fastening applications provide confined working channels and spaces and often must be fed through an endoscope to permit visualization or other small lumen guide catheters to the place where the fasteners are to be deployed.

Visualization under these conditions is difficult and may be obscured. Hence, deploying fasteners in such environments is difficult. It is particularly difficult to visually measure distance, which is so important when deploying a device designed to attach to tissue. Further, most often, more than one fastener is required. It would be desirable to be able to deploy fasteners with a procedure that is uniform in application and repeatable. The present invention addresses these and other issues.

### SUMMARY

The invention provides an assembly comprising a stylet that guides a fastener into tissue, the stylet having a proximal end, a bolt attached to the proximal end of the stylet, and a receiver that slidingly receives the bolt permitting linear movement of the bolt and stylet along a path. The assembly may further comprise a pusher that intersects the path of the stylet and pushes the fastener along the stylet.

The pusher may be carried on the stylet distal to where the pusher intersects the path of the stylet. The pusher may be tubular and include an opening permitting the pusher to be received on the stylet.

The bolt may include a lumen that slidingly receives the pusher. The pusher may intersect the path at an intersection and the assembly may further include a loading station that permits a fastener to be loaded onto the stylet distal to the intersection.

The loading station may have a given length dimension and the assembly may further comprise a fastener loader. The fastener loader may have a width dimension less than the given length dimension for loading a fastener onto the stylet within the loading station. The fastener loader is preferably arranged to carry a plurality of the fasteners.

The bolt may include a projecting handle and the receiver may include a track that receives the handle and restricts movement of the bolt. The track may include at least one transverse slot that receive the bolt handle and locks the bolt in a predetermined longitudinal position.

The invention further provides an assembly comprising first and second subassemblies. The first subassembly includes a first stylet that guides a first fastener into tissue and has a proximal end, a first bolt attached to the proximal end of the first stylet, and a first receiver that slidingly receives the first bolt permitting linear movement of the first bolt and stylet along a first path into the tissue. The second subassembly includes a second stylet that guides a second fastener into the tissue and has a proximal end, a second bolt attached to the proximal end of the second stylet, and a second receiver that slidingly receives the second bolt permitting linear movement of the second bolt and stylet along a second path into the tissue. The first and second subassemblies are carried by a common housing.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like reference numerals identify like elements, and wherein:

FIG. 1 is a front cross-sectional view of the esophageal-gastro-intestinal tract from a lower portion of the esophagus to the duodenum;

FIG. 2 is a front cross-sectional view of the esophageal-gastro-intestinal tract illustrating a Grade I normal appearance movable flap of the gastroesophageal flap valve (in dashed lines) and a Grade III reflux appearance gastroesophageal flap of the gastroesophageal flap valve (in solid lines);

FIG. 3 is a side view of an apparatus for restoring the flap of a GEFV according to an embodiment of the invention;

FIG. 4 is a side view similar to FIG. 3 showing stomach tissue being molded and ready to receive one or more fasteners;

FIG. 5 is a perspective view of a fastener that may be used in an assembly according to an embodiment of the invention;

FIG. 6 is a side view of the fastener of FIG. 5;

FIG. 7 is a perspective view with portions cut away of a fastener assembly according to an embodiment of the invention in an early stage of deploying the fastener of FIGS. 5 and 6;

FIG. 8 is a perspective view of the assembly of FIG. 7 shown with the fastener in an intermediate stage of deployment;

FIG. 9 is a perspective view of the assembly of FIG. 7 shown with the fastener almost completely deployed;

FIG. 10 is a perspective view showing the fastener of the assembly of FIG. 7 fully deployed and securely fastening a pair of tissue layers together;

FIG. 11 is a perspective view of a fastener delivery and deployment assembly according to an embodiment of the invention;

FIG. 12 is a perspective view with portions cut away of the assembly of FIG. 11 illustrating inner structure thereof; and

FIG. 13 is a perspective view of a fastener loader loading a fastener onto the assembly of FIGS. 11 and 12 according to an embodiment of the present invention.

### DETAILED DESCRIPTION

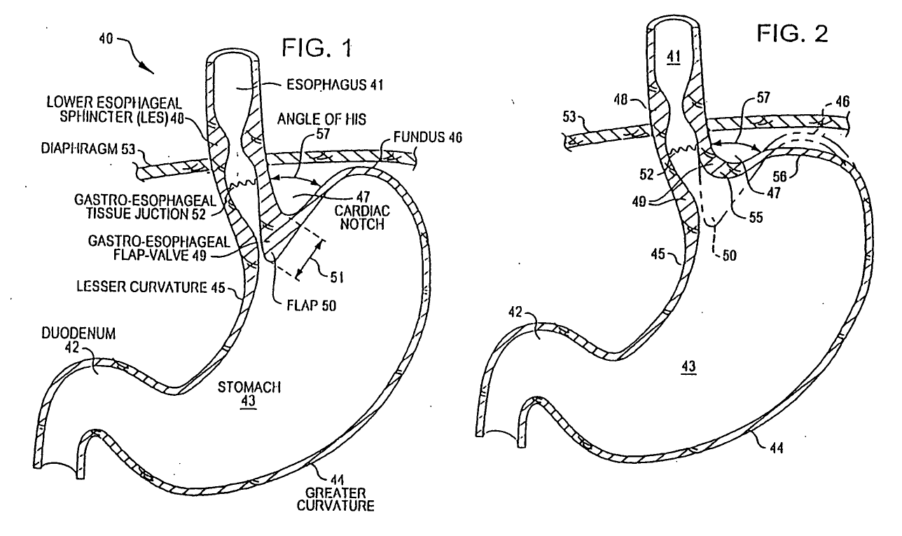

FIG. 1 is a front cross-sectional view of the esophageal-gastro-intestinal tract 40 from a lower portion of the esophagus 41 to the duodenum 42. The stomach 43 is characterized by the greater curvature 44 on the anatomical left side and the lesser curvature 45 on the anatomical right side. The tissue of the outer surfaces of those curvatures is referred to in the art as serosa tissue. As will be seen subsequently, the nature of the serosa tissue is used to advantage for its ability to bond to like serosa tissue.

The fundus 46 of the greater curvature 44 forms the superior portion of the stomach 43, and traps gas and air bubbles for burping. The esophageal tract 41 enters the stomach 43 at an esophageal orifice below the superior portion of the fundus 46, forming a cardiac notch 47 and an acute angle with respect to the fundus 46 known as the Angle of His 57. The lower esophageal sphincter (LES) 48 is a discriminating sphincter able to distinguish between burping gas, liquids, and solids, and works in conjunction with the fundus 46 to burp. The gastroesophageal flap valve (GEFV) 49 includes a moveable portion and an opposing more stationary portion.

The moveable portion of the GEFV 49 is an approximately 180degree, semicircular, gastroesophageal flap 50 (alternatively referred to as a “normal moveable flap” or “moveable flap”) formed of tissue at the intersection between the esophagus 41 and the stomach 43. The opposing more stationary portion of the GEFV 49 comprises a portion of the lesser curvature 45 of the stomach 43 adjacent to its junction with the esophagus 41. The gastroesophageal flap 50 of the GEFV 49 principally comprises tissue adjacent to the fundus 46 portion of the stomach 43. It is about 4 to 5 cm long (51) at it longest portion, and its length may taper at its anterior and posterior ends.

The gastroesophageal flap 50 is partially held against the lesser curvature 45 portion of the stomach 43 by the pressure differential between the stomach 43 and the thorax, and partially by the resiliency and the anatomical structure of the GEFV 49, thus providing the valving function. The GEFV 49 is similar to a flutter valve, with the gastroesophageal flap 50 being flexible and closeable against the other more stationary side.

The esophageal tract is controlled by an upper esophageal sphincter (UES)in the neck near the mouth for swallowing, and by the LES 48 and the GEFV 49 at the stomach. The normal anti-reflux barrier is primarily formed by the LES 48 and the GEFV 49 acting in concert to allow food and liquid to enter the stomach, and to considerably resist reflux of stomach contents into the esophagus 41 past the gastroesophageal tissue junction 52. Tissue aboral of the gastroesophageal tissue junction 52 is generally considered part of the stomach because the tissue protected from stomach acid by its own protective mechanisms. Tissue oral of the gastroesophageal junction 52 is generally considered part of the esophagus and it is not protected from injury by prolonged exposure to stomach acid. At the gastroesophageal junction 52, the juncture of the stomach and esophageal tissues form a zigzag line, which is sometimes referred to as the “Z-line”. For the purposes of these specifications, including the claims, “stomach” means the tissue aboral of the gastroesophageal junction 52.

FIG. 2 is a front cross-sectional view of an esophageal-gastro-intestinal tract illustrating a Grade I normal appearance movable flap 50 of the GEFV 49 (shown in dashed lines) and a deteriorated Grade III gastroesophageal flap 55 of the GEFV 49 (shown in solid lines). As previously mentioned, a principal reason for regurgitation associated with GERD is the mechanical failure of the deteriorated (or reflux appearance) gastroesophageal flap 55 of the GEFV 49 to close and seal against the higher pressure in the stomach. Due to reasons including lifestyle, a Grade I normal gastroesophageal flap 50 of the GEFV 49 may deteriorate into a Grade III deteriorated gastroesophageal flap 55. The anatomical results of the deterioration include moving a portion of the esophagus 41 that includes the gastroesophageal junction 52 and LES 48 toward the mouth, straightening of the cardiac notch 47, and increasing the Angle of His 57. This effectively reshapes the anatomy aboral of the gastroesophageal junction 52 and forms a flattened fundus 56.

The deteriorated gastroesophageal flap 55 shown in FIG. 2 has a gastroesophageal flap valve 49 and cardiac notch 47 that are both significantly degraded. Dr. Hill and colleagues developed a grading system to describe the appearance of the GEFV and the likelihood that a patient will experience chronic acid reflux. L. D. Hill, et al., The gastroesophageal flap valve: in vitro and in vivo observations, Gastrointestinal Endoscopy 1996:44:541-547. Under Dr. Hill's grading system, the normal movable flap 50 of the GEFV 49 illustrates a Grade I flap valve that is the least likely to experience reflux. The deteriorated gastroesophageal flap 55 of the GEFV 49 illustrates a Grade III (almost Grade IV) flap valve. A Grade IV flap valve is the most likely to experience reflux. Grades II and III reflect intermediate grades of deterioration and, as in the case of III, a high likelihood of experiencing reflux. With the deteriorated GEFV represented by deteriorated gastroesophageal flap 55 and the fundus 46 moved inferior, the stomach contents are presented a funnel-like opening directing the contents into the esophagus 41 and the greatest likelihood of experiencing reflux. Disclosed subsequently is a device, assembly, and method which may be employed to advantage according to an embodiment of the invention in restoring the normal gastroesophageal flap valve anatomy.

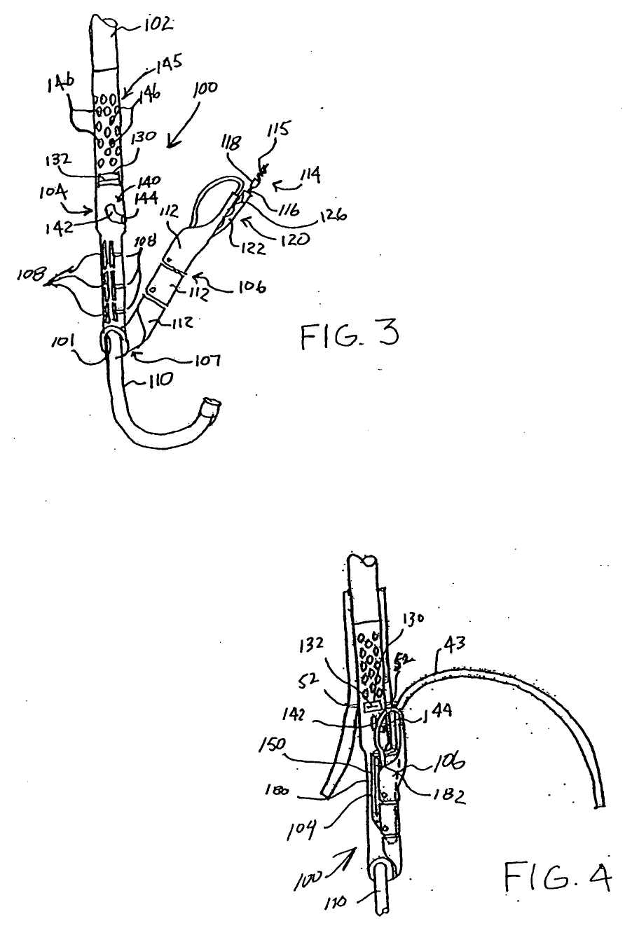

Referring now to FIG. 3, it shows a device 100 according to an embodiment of the present invention. The device 100 includes a longitudinal member 102 for transoral placement of the device 100 into the stomach. Hence, the device 100 is at the distal end of the longitudinal member 102. Located at the proximal end of the longitudinal member is a control assembly to be described subsequently in detail in connection with fastener delivery and deployment to maintain the restored GEFV flap.

The device further includes a first member 104, hereinafter referred to as the chassis, and a second member 106, hereinafter referred to as the bail. The chassis 104 and bail are hingedly coupled at 107. The chassis 104 and bail 106 form a tissue shaper which, as described subsequently in accordance with this embodiment of the present invention, shapes tissue of the stomach into the flap of a restored gastroesophageal flap valve. The chassis 104 and bail 106 are carried at the distal end of the longitudinal member 102 for placement in the stomach.

The device 100 has a longitudinal passage 101 to permit an endoscope 110 to be guided through the device and into the stomach. This permits the endoscope to service as a guide for guiding the device 100 through the patient's throat, down the esophagus, and into the stomach. It also permits the gastroesophageal flap valve restoration procedure to be viewed at each stage of the procedure.

To facilitate shaping of the stomach tissue, the stomach tissue is drawn in between the chassis 104 and the bail 106. Further, to enable a flap of sufficient length to be formed to function as the flap of a gastroesophageal flap valve, the stomach tissue is pulled down so that the fold line is substantially juxtaposed to the opening of the esophagus into the stomach. Hence, the stomach is first gripped at a point out and away from the esophagus and the grip point is pulled to almost the hinged connection 107 of the chassis 104 and bail 106. As described in copending application Ser. No. 11/001,666, filed Nov. 30, 2004, entitled FLEXIBLE TRANSORAL ENDOSCOPIC GASTROESOPHAGEAL FLAP VALVE RESTORATION DEVICE AND METHOD, which application is incorporated herein by reference, the device 100 is fed down the esophagus with the bail 106 substantially in line with the chassis 104. To negotiate the bend of the throat, and as described in the aforementioned referenced application, the chassis 104 and bail 106 are rendered flexible. The chassis 104 is rendered flexible by the slots 108 and the bail 106 is rendered flexible by the hingedly coupled links 112. Further details concerning the flexibility of the chassis 104 and the bail 106 may be found in the aforementioned referenced application.

As further shown in FIG. 3, the device includes a tissue gripper 114. The gripper 114, in this embodiment, comprises a helical coil 115. The coil 115 is carried at the end of a cable 116 and may be attached to the end of the cable or be formed from the cable. In this embodiment, the helical coil 115 is attached to the cable 116 and is preceded by a guide 118 whose function will be described subsequently.

The helical coil 115 is shown in an approximate position to engage the stomach tissue out and away from the opening of the esophagus to the stomach. The helical coil 115 is guided into position by a guide structure 120 carried on the bail 106. The guide structure 120 comprises a guide tube 122. When the device 100 is first introduced down the esophagus into the stomach, the helical coil 115 is caused to reside well within the guide tube 122 to preclude the helical coil from accidentally or inadvertently snagging esophageal or stomach tissue.

The guide tube includes a longitudinal slit 126 having a circuitous configuration. The slit 126 permits the end of the cable to release or disassociate from the bail after the stomach tissue is gripped. The circuitous configuration of the slit 126 assures confinement of the cable 116 within the guide tube 122 until release of the cable is desired. The proximal end of the slit 126 has an enlarged portion or opening (not shown). This opening permits the cable and helical coil to reenter the lumen when the device 100 is readied for a repeated stomach tissue shaping procedure. To that end, the guide 118 has a conical surface that serves to guide the cable end back into the opening of the slit 126.

With continued reference to FIG. 3, the device 100 further comprises a fastener deployer 140. The fastener deployer includes at least one fastener deployment guide 142. The fastener deployment guide 142 takes the form of a guide lumen. Although only one guide lumen 142 is shown, it will be appreciated that the device 100 may include a plurality of such lumens without departing from the invention. The guide lumen terminates at a delivery point 144 where a fastener is driven into the molded stomach tissue. The fastener deployer may take the form of any one of the assemblies fully described and claimed, for example, in

The device 100 further includes a window 130 within the chassis 104. The window is formed of a transparent or semitransparent material. This permits gastroesophageal anatomy, and more importantly the gastroesophageal junction (Z-line) to be viewed with the endoscope 110. The window includes a location marker 132 which has a know position relative to the fastener delivery point 144. Hence, by aligning the marker with a known anatomical structure, the fastener will be delivered a known distance from or at a location having a predetermined relation to the marker. For example, by aligning the marker with the Z-line, it will be know that the fastener will be placed aboral of the Z-line and that serosa tissue will be fastened to serosa tissue. As previously mentioned, this has many attendant benefits.

It may also be mentioned at this point that the device 100 further includes an invaginator 145 including a plurality of orifices 146. These orifices 146, which alternatively may be employed on the longitudinal member 102, are used to pull a vacuum to cause the device 100 to grip the inner surface of the esophagus. This will serve to stabilize the esophagus and maintain device positioning during the procedure. This vacuum gripping of the esophagus may also be used to particular advantage if the patient suffers from a hiatal hernia. Upon being thus gripped, the esophagus may be moved downwardly with the device toward the stomach to eliminate the hiatal hernia.

Referring now to FIG. 4, here the bail 106 is now closed and stomach tissue aboral of the Z-line 52 is confined between the bail 106 and chassis 104 to create a fold 150. The fold is also adjacent the fastener delivery point 144 at the end of the fastener guide lumen. Since the fastener deployment point 144 is a known predetermined distance from the marker 132 of the window 130, and since the marker 132 is aligned with the Z-line 52, when a fastener is delivered from the fastener deployer of the device, the fastener will exit the fastener delivery point 144 at a point known to be aboral of the Z-line 52. This assures that only serosa tissue is being adhered to serosa tissue in the fixation of the stomach tissue in creating the flap 150. The flap 150 comprises layers 180 and 182 of stomach tissue.

With the tissue layers 180 and 182 now disposed within the mold of the chassis 104 and bail 106, the bail 106 may now be locked with respect to the chassis 104. It is now time to fasten the tissue layers 180 and 182 together by ejecting a fastener from the fastener deployer lumen 142 at the fastener delivery point 144.

Before a fastener is ejected from the fastener deployer lumen 142, the stomach may be inflated through the endoscope 110. The stomach may be inflated to a point where one has a good view of the tissue fold and bail 106 with the endoscope.

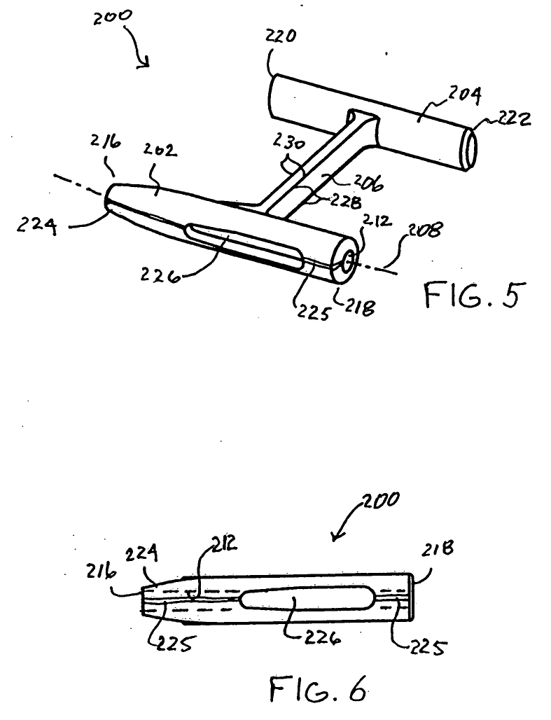

FIG. 5 is a perspective view and FIG. 6 is a side view of a fastener 200 according to an embodiment of the present invention. The fastener 200 generally includes a first member 202, a second member 204, and a connecting member 206. As may be noted in FIG. 3, the first member 202 and second member 204 are substantially parallel to each other and substantially perpendicular to the connecting member 206 which connects the first member 202 to the second member 204.

The first member 202 is generally cylindrical or can have any other shape. It has a longitudinal axis 208 and a through channel 212 along the longitudinal axis 208. The through channel 212 is formed by a through bore which is dimensioned to be slidingly received on a tissue piercing deployment wire to be described.

The first member 202 also includes a first end 216 and a second end 218. Similarly, the second member 204 includes a first end 220 and a second end 222. The first end 216 of member 202 forms a pointed dilation tip 224. The dilation tip 224 may be conical and more particularly takes the shape of a truncated cone. The tip can also be shaped to have a cutting edge in order to reduce tissue resistance.

The first and second members 202 and 204 and the connecting member 206 may be formed of different materials and have different textures. These materials may include, for example, plastic materials such as polypropylene, polyethylene, polyglycolic acid, polyurethane, or a thermoplastic elastomer. The plastic materials may include a pigment contrasting with body tissue color to enable better visualization of the fastener during its deployment. Alternatively, the fastener may be formed of a metal, such as stainless steel or a shape memory metal, such as Nitinol.

As may be further noted in FIG. 5, the connecting member 206 has a vertical dimension 228 and a horizontal dimension 230 which is transverse to the vertical dimension. The horizontal dimension is substantially less than the vertical dimension to render the connecting member 206 readily bendable in a horizontal plane. The connecting member is further rendered bendable by the nature of the material from which the fastener 200 is formed. The connecting member may be formed from either an elastic plastic or a permanently deformable plastic. An elastic material would prevent compression necrosis in some applications.

It may be noted in FIGS. 5 and 6, that the first member 202 has a continuous lengthwise slit 225 extending between the first and second ends 216 and 218. The slit 225 includes an optional slot portion 226 that communications with the through channel 212. The slot 226 has a transverse dimension for more readily enabling receipt of a tissue piercing deployment wire during deployment of the fastener 200. Also, because the fastener number 202 is formed of flexible material, the slit 225 may be made larger through separation to allow the deployment wire to be snapped into and released from the through channel 212. This permits both ready fastener loading and fastener release from the deployment wire after deployment in the tissue layers. The slit 225 extends substantially parallel to the through channel 212 and the center axis 208 of the first member 202. It may also be noted that the slit 225 has a width dimension that is smaller or less than the diameter D of the through channel 212. This assures that the fastener 200 will remain on a tissue piercing deployment wire as it is pushed towards and into the tissue as will be seen subsequently.

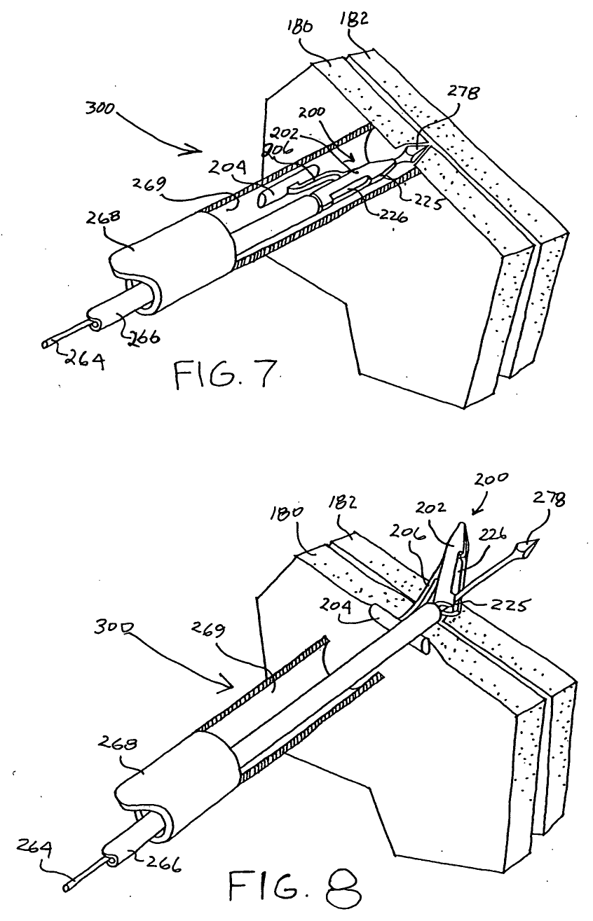

Referring now to FIGS. 7-10, they are perspective views with portions cut away of a fastener assembly 300 illustrating a manner in which a fastener may be deployed according to an embodiment of the present invention. The tissue layer portions above the fastener 200 have been shown cut away in FIGS. 7-10 to enable the deployment procedure to be seen more clearly. The assembly 300 generally includes the fastener 200, a deployment wire 264, a pusher 266, and a guide tube 268 having a guide lumen 269 (analogous to guide 142 of FIGS. 3 and 4).

As will be noted in FIG. 7, the first member 202 of the fastener 200 is slidingly received on the deployment wire 264. The deployment wire 264 has a pointed tip 278 for piercing the tissue layers 180 and 182 to be fastened together. The tip 278 cuts sufficient tissue to enable the fastener member 202 to readily pass through the tissue layers 180 and 182. It may also serve as a guide to guide the wire 264 off of the member 202 at the end of the deployment. The tissue piercing wire 264, fastener 200, and the pusher 266 are all within the guide tube 268. The guide tube 268 may preferably take the form of a guide channel, such the guide 142 of FIG. 3 and 4, as previously mentioned.

As will also be further noted in FIG. 7, the second member 204 is disposed along side the first member 202. This is rendered possible by the flexibility of the connecting member 206 and aids in proper deployment of the fastener 200.

The subassembly of the tissue piercing wire 264, fastener 200, and pusher 266 are guided to the intended deployment location by the guide tube 268. With the first member 202 of the fastener 200 slidingly received on the tissue piercing wire 264 and with the pusher 266 just touching the first member 202 on the tissue piercing wire 264, the tissue piercing wire 264 is advanced a controlled distance to cause the tip 278 to pierce through the tissue layers 180 and 182 a control distance.

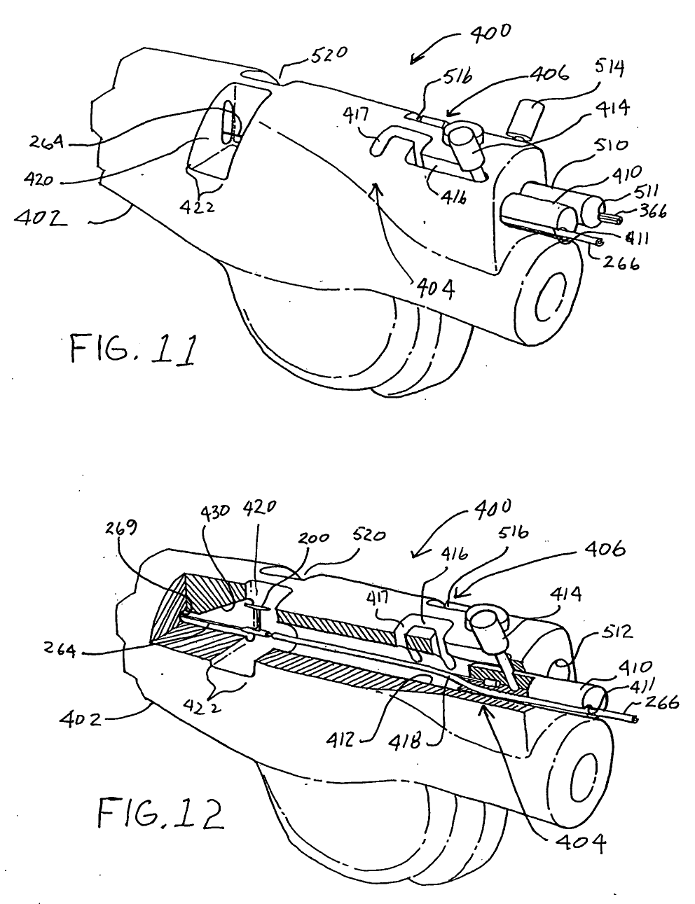

As shown in FIG. 8, the tissue piercing wire 264 has been advanced a controlled distance by the assembly of FIGS. 11 and 12 as described subsequently to pierce the tissue layers 180 and 182. The pusher 266 is then used to push the first member 202 of the fastener 200 through the tissue layers 180 and 182 on the tissue piercing wire 264.

As may be further seen in FIG. 8, the first member 202 has been pushed forward by the pusher 266 to cause the second member 204 to engage the tissue layer 180. Continued pushing of the first member 202 causes the first member to pivot in a counter clockwise direction because the second member 204 is held by the tissue layer 180. The counter clockwise movement of the first member 202 causes the wire 264 to spread the slit 225 open, to pass down the slit to enter slot portion 226 and to eventually pass through the slit 225 at end 218. The fastener 200 is then ready to release from the wire 264.

In FIG. 9, it will now be seen that the second end 218 of the first member 202 has cleared the wire 264 and tissue layer 182. The tissue piercing wire 264 may now be retracted into the pusher 266 and the tissue piercing wire 264 and pusher 266 may be withdrawn.

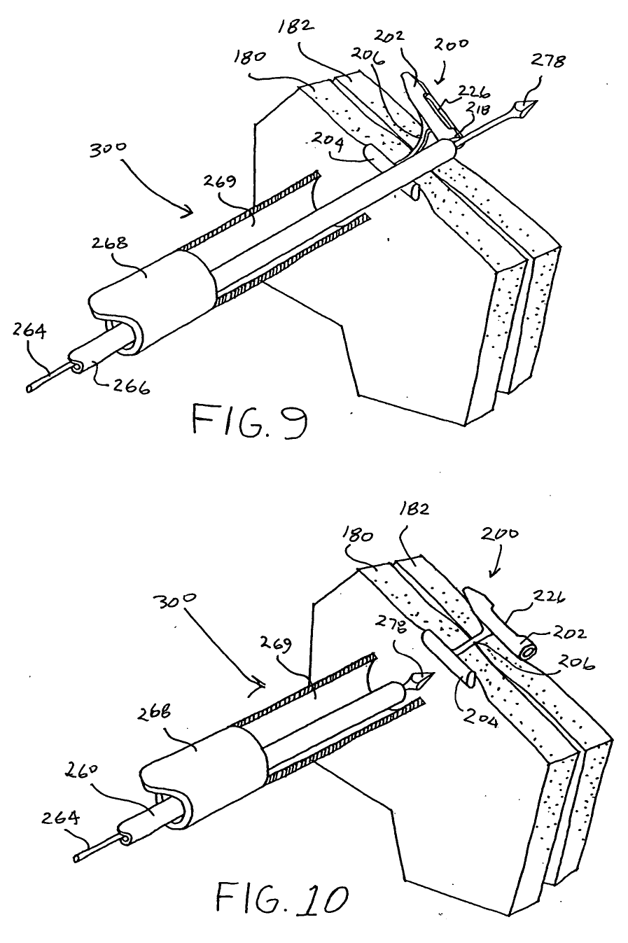

FIG. 10 illustrates the fastener 200 in its fully deployed position. It will be noted that the fastener has returned to its original shape. The tissue layers 180 and 182 are fastened together between the first member 202 of the fastener 200 and the second member 204 of the fastener 200. The connecting member 106 extends through the tissue layers 180 and 182.

Referring now to FIGS. 11 and 12, FIGS. 11 and 12 illustrate a control assembly 400 for controlling the delivery and deployment of fasteners according to an embodiment of the present invention. More specifically, the assembly 400, according to this embodiment, is located at the proximal end of the longitudinal member 102 (FIG. 3) for deploying fasteners into stomach tissue in a manner as described with respect to FIGS. 5-10 for maintaining the manipulated stomach tissue which has been folded and molded to restore a GEFV flap.

The assembly of FIGS. 11 and 12 will be described with particular reference to fastener delivery and deployment. The assembly 400 generally includes a housing 402. The housing includes identical, side-bi-side control assemblies 404 and 406. Since the control assemblies 404 and 406 are identical, only assembly 404 will be described in detail herein.

The assembly 404 includes a bolt 410, a receiver 412 that slidingly receives the bolt 410 and the pusher 266. Projecting from the bolt is a handle 414. The handle extends through a track 416 in the housing 402 and restricts and measures the movement of the bolt 410.

As previously mentioned, the control assemblies 404 and 406 are side-bi-side and identical. Hence, the assembly 406 may also be seen to include a bolt 510, a pusher 366, a receiver 512, and a handle 514 projecting through a track 516. The operation of the assembly 406 is identical to the operation of the assembly 404 to be described subsequently.

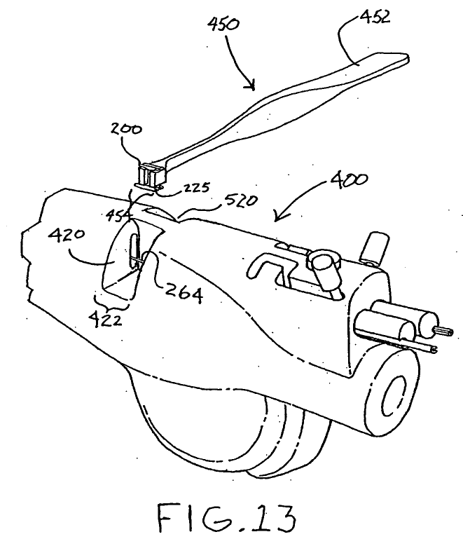

The assembly 404 still further includes a fastener loading station 420. The loading station 420 has a length dimension 422 sufficient to receive a fastener loader to be described subsequently with respect to FIGS. 13 and 14. The fastener loader and loading station facilitate loading of fasteners onto the deployment stylet 264. The assembly 406 also includes such a loading station 520.

As may be best seen in FIG. 12, the bolt 410 of assembly 404 is attached to the proximal end of the stylet 264. Hence, the bolt and stylet are arranged for linear movement when the bolt 410 is moved within the receiver 412 with the handle 414 along the track 416.

The pusher 264 intersects the path of the stylet 264 at an intersection point 418. The pusher, as best described in copending application Ser. No. 11/043,903, includes an opening at the intersection 418. The opening permits the stylet to be fed into the pusher and hence to allow the pusher 266 to be carried by the stylet 264 distal to the intersection 418. As previously seen, this permits the pusher 266 to engage the fastener 200. Also, the loading station 420 is distal to the intersection 418 to permit the fastener 200 to be loaded onto the stylet 264 and engaged by the pusher 266.

The bolt 410 further includes a lumen 411 that slidingly receives the pusher 266. This permits the movement of the pusher 266 to be controlled independently of the movement of the bolt 410 and the stylet 264. The bolt 510 also includes such a lumen 511 as may be seen in FIG. 11.

As may be further noted in FIG. 12, the assembly 404 further includes a funnel shaped wall 430 between the loading station 420 and the guide lumen 269. As may be recalled from FIGS. 7-10, the guide lumen 269 guides the stylet 264, fastener 200, and pusher 266 to the desired location for deploying the fasteners. The funnel shaped wall 430, as more fully described in copending application Ser. No. (2234-3-6), incorporated herein by reference, serves to preposition the second member 204 of the fastener 200 within the guide lumen 269 as best seen in FIG. 7. The second member 204 is prepositioned as a trailing member along side the first member 202 with the connecting member 206 therebetween. This fastener configuration and prepositioning assists in the proper functioning of the second member 204 as the fastener 200 is deployed. The second member 204 is automatically rendered in its preposition along side the first member 202 with the connecting member 206 therebetween as the fastener 200 is translated distally through the funnel shaped wall section towards the guide lumen 269.

In operation, when it is time to advance the stylet 264 in through the tissue as shown in FIG. 8, the handle 414 is moved in a distal direction forcing the stylet 264 to move distally. The handle and thus the stylet movement is restricted and measured by a transverse portion of slot 417 of the track 416. The handle 414 may be locked in a longitudinal position within the transverse portion 417 of the track. The fastener 200 is advanced by the pusher 266. After a fastener is deployed, the distal end of the pusher is drawn back to be proximal to the loading station 420 to permit another fastener to be loaded onto the stylet 264.

The fasteners are loaded onto the stylet by presenting the slit 225 of the fasteners to the stylet. The slit 225 (FIG. 6) is widened by the stylet 264 and the stylet 264 slips through the slit 225 and into the through channel 212 of the fastener first member 202.

FIG. 13 shows a fastener loader 450 which may be employed for loading the fasteners onto the stylet. The loader 450 has a handle 452 permitting it to be readily hand-holdable. At the distal end, the loader is arranged to carry a plurality of fasteners 200. The loader presents the fasteners so that the slit 225 will be aligned with the stylet 264.

The holder has a width dimension 454 that is less than the length dimension 422 (FIGS. 11 and 12) of the loading station 420. Hence, the loader 450 may be inserted into a loading station for mounting a fastener onto a corresponding stylet. The loader 450 may be used on either side of the assembly 400 for loading a fastener onto stylet 264 at loading station 420 or loading a fastener onto stylet 364 at loading station 520.

While particular embodiments of the present invention have been shown and described, modifications may be made, and it is therefore intended in the appended claims to cover all such changes and modifications which fall within the true spirit and scope of the invention.

What is claimed is:

1. An assembly comprising:
a stylet that guides a fastener into tissue, the stylet having a proximal end;
a bolt attached to the proximal end of the stylet;
a receiver that slidingly receives the bolt permitting linear movement of the bolt and stylet along a path into the tissue.

2. The assembly of claim 1 further comprising a pusher that intersects the path of the stylet and pushes the fastener along the stylet.

3. The assembly of claim 2, wherein the pusher is carried on the stylet distal to where the pusher intersects the path of the stylet.

4. The assembly of claim 2, wherein the bolt includes a lumen that slidingly receives the pusher.

5. The assembly of claim 2, wherein the pusher intersects the path at an intersection and wherein the assembly further includes a loading station that permits a fastener to be loaded onto the stylet, the loading station being distal to the intersection.

6. The assembly of claim 2, wherein the pusher is tubular and includes an opening permitting the pusher to be received on the stylet.

7. The assembly of claim 1, further comprising a loading station that permits a fastener to be loaded onto the stylet and having a given length dimension and a fastener loader, the fastener loader having a width dimension less than the given length dimension for loading a fastener onto the stylet within the loading station.

8. The assembly of claim 7, wherein the fastener loader is arranged to carry a plurality of the fasteners.

9. The assembly of claim 1, wherein the bolt includes a projecting handle and wherein the receiver includes a track that receives the handle and restricts movement of the bolt.

10. The assembly of claim 9, wherein the track includes at least one transverse slot that receive the bolt handle and locks the bolt in a predetermined longitudinal position.

11. An assembly comprising:
a first subassembly including a first stylet that guides a first fastener into tissue and having a proximal end, a first bolt attached to the proximal end of the first stylet, a first receiver that slidingly receives the first bolt permitting linear movement of the first bolt and stylet along a first path into the tissue; and
a second subassembly including a second stylet that guides a second fastener into the tissue and having a proximal end, a second bolt attached to the proximal end of the second stylet, a second receiver that slidingly receives the second bolt permitting linear movement of the second bolt and stylet along a second path into the tissue,
the first and second subassemblies being carried by a common housing.

12. The assembly of claim 10, wherein the first and second subassemblies are arranged side-by-side on the common housing.

13. The assembly of claim 11 wherein the first subassembly comprises a first pusher that intersects the first path of the first stylet and pushes the first fastener along the first stylet and wherein the second subassembly comprises a second pusher that intersects the second path of the second stylet and pushes the second fastener along the second path.

14. The assembly of claim 13, wherein the first and second pushers are carried on the first and second stylets distal to where the first and second pushers intersect the first and second paths of the first and second stylets, respectively.

15. The assembly of claim 13, wherein the first and second bolts include first and second lumens that slidingly receive the first and second pushers, respectively.

16. The assembly of claim 13, wherein the first and second pushers intersect the first and second paths at first and second intersections, respectively, and wherein the assembly further includes first and second loading stations that permit the first and second fasteners to be loaded onto the first and second stylets, the first and second loading stations being distal to the first and second intersections.

17. The assembly of claim 16, wherein each of the loading stations has a given length dimension and wherein the assembly further comprises a fastener loader, the fastener loader having a width dimension less than the given length dimension for loading a fastener onto the first and second stylets within the first and second loading stations

18. The assembly of claim 17, wherein the fastener loader is arranged to carry a plurality of the fasteners.

19. The assembly of claim 13, wherein the first and second pushers are tubular and wherein each of the first and second pushers includes an opening permitting the first pusher to be received on the first stylet and the second pusher to be received on the second stylet.

20. The assembly of claim 11, wherein the first and second bolts each includes a projecting handle and wherein the first and second receivers each includes a track that receives the handle and restricts movement of its respective bolt.

21. The assembly of claim 20, wherein each track includes at least one transverse slot that receive its respective bolt handle and locks its respective bolt in a predetermined longitudinal position.
